(19) United States
(12) Patent Application Publication
(10) Pub. No.: US 2022/0249720 A1
(43) Pub. Date: Aug. 11, 2022

(54) REPROCESSOR HAVING A VARIABLE ORIFICE DEVICE

(71) Applicant: ASP Global Manufacturing GmbH, Shaffhausen (CH)

(72) Inventor: Ujjal BHAUMIK, Yorba Linda, CA (US)

(21) Appl. No.: 17/618,805

(22) PCT Filed: Jun. 16, 2020
(86) PCT No.: PCT/IB2020/055616

§ 371 Date: Dec. 13, 2021

Related U.S. Application Data
(60) Provisional application No. 62/864,183, filed on Jun. 20, 2019.

(51) Int. Cl. A61L 2/18; A61L 2/24

(52) U.S. Cl. CPC A61L 2/18; A61L 2/24; A61L 2202/24; A61L 2202/15; A61L 2202/14

(57) ABSTRACT

A decontamination system suitable for cleaning a medical device having lumens of varying diameters that join together may include a variable-orifice devices (e.g., air proportional valves). A control system may be configured to adjust the variable-orifice devices based on flow characteristics, e.g., flow rates, such that the flow rates in the medical-device lumens are similar.

### FIELD

The subject matter disclosed herein relates to decontaminating instruments having lumens, particularly medical devices, such as endoscopes.

### BACKGROUND

Endoscopes are reusable medical devices. An endoscope should be reprocessed, i.e., decontaminated, between medical procedures in which it is used to avoid causing infection or illness in a subject. Endoscopes are difficult to decontaminate as has been documented in various news stories. See, e.g., Chad Terhune, “Superbug outbreak: UCLA will test new scope-cleaning machine,” LA Times, Jul. 22, 2015, http://www.latimes.com/business/la-fi-ucla-superbug-scope-testing-20150722-story.html (last visited Oct. 30, 2017). Endoscope reprocessing may be conducted by a healthcare worker, or with the assistance of machinery, such as an endoscope reprocessor, e.g., the EVOTECH® Endoscope Cleaner and Reprocessor of Advanced Sterilization Products, Inc.

Two endoscope reprocessing methods pertinent to the present disclosed subject matter include disinfection or liquid-chemical sterilization. Both types of procedures may include steps of removing foreign material from the endoscope, cleaning the endoscope, introducing a disinfectant solution or a liquid-chemical sterilant to the endoscope, rinsing the endoscope, and drying the endoscope.

### SUMMARY OF THE DISCLOSURE

Disclosed herein is a decontamination system suitable for cleaning a medical device having lumens of varying diameters that merge together. The disinfection system may include a source of a first decontamination fluid, a gas compressor, a first variable-orifice device, and a second variable-orifice device. The variable orifice devices may be, e.g., air proportional valves, such as a closed loop air proportional valves. The disinfection system may also include a first flush line connected to the gas compressor via the first variable-orifice device and further connected to the source of the first decontamination fluid. The disinfection system may also include a second flush line connected to the gas compressor via the second variable-orifice device and further connected to the source of the first decontamination fluid. Additionally, the first flush line may include a first output and the second flush line may include a second output. The first output and the second output may be connected to lumens of an endoscope, e.g., the suction channel, the biopsy channel, the air channel, and the water channel. In many endoscopes, the suction channel and the biopsy channel merge to form a joined channel, often referred to as a suction/biopsy channel. Similarly, the air channel and the water channel merge to form a joined channel, often referred to as an air/water channel.

The first variable-orifice device may include a first orifice having a first size and the second variable-orifice device may include a second orifice having a second size that is different than the first size. A first flow-characteristic sensor may be disposed along the first flush line between the first variable-orifice device and the first output. A second flow-characteristic sensor may be disposed along the second flush line between the first variable-orifice device and the second output. The flow-characteristic sensors may be, e.g., flow sensors.

The decontamination system may also comprise a first source for containing decontamination fluids. This first source may be disposed between the first variable-orifice device and the first flush line. The decontamination system may also comprise a second source for containing the decontamination fluids. This second source may be disposed between the second first variable-orifice device and the second flush line.

The decontamination system may also include a first bypass line connected to the first variable-orifice device and the first flush line. The decontamination system may also include a second bypass line connected to the second variable-orifice device and the second flush line.

A first valve may be connected to the first variable-orifice device, the first flush line, and the first bypass line. This first valve may direct air from the first variable-orifice device toward the first source or toward the bypass line. A second valve may be connected to the second variable-orifice device, the second flush line, and the second bypass line. This second valve may direct air from the first variable-orifice device toward the first source or toward the bypass line.

The decontamination system may also comprise a control system that is connected to the first flow-characteristic sensor, the first closed-loop air-proportional valve, the second flow-characteristic sensor and the second closed-loop air-proportional valve. The control system may be configured to adjust the first size of the first orifice or the second size of the second orifice based on a first output from the first flow-characteristic sensor, a second output from the second flow-characteristic sensor, or both.

The decontamination system may be operated according to the following steps. A first volume of gas, e.g., air, may be flowed through the first variable-orifice device and into the first flush line, which contains the decontamination liquid. A second volume of the gas may be flowed through a second variable-orifice device and into a second flush line, which also contains the decontamination liquid. A first channel of an endoscope may be connected to a first output of the first flush line and a second channel of an endoscope may be connected to a second output of the second flush line. As such, a first volume of the gas may be flowed through the first channel, a second volume of the gas may be flowed through the second channel, and the first volume of gas and the second volume of the gas may be flowed through the joined channel. Furthermore, a flow characteristic (e.g., flow rate) along the first flush line or the second flush line may be measured with the flow-characteristic sensor. Based on the flow characteristic, the first size of the first orifice, the second size of the second orifice, or both, may be changed. Preferably, the first size, second size, or both are changed to cause the first flow rate and the second flow rate to become approximately equal.

These first and second sizes may be changed by the controller. As such, the method may also include receiving at the controller the flow characteristic, determining, with the controller, whether the flow characteristic should be changed, sending an instruction to the first variable-orifice, the second variable-orifice device, or both, to change the size of the first orifice, the second orifice, or both; and changing the size of the first orifice, the second orifice, or both.

The method may result in purging the liquid from the endoscope's channels and drying the endoscope channels.

### BRIEF DESCRIPTION OF THE DRAWINGS

While the specification concludes with claims, which particularly point out and distinctly claim the subject matter described herein, it is believed the subject matter will be better understood from the following description of certain examples taken in conjunction with the accompanying drawings, in which like reference numerals identify the same elements and in which:

FIG. 1 depicts a front elevational view of an exemplary reprocessing system;

FIG. 2 depicts a schematic diagram of a single decontamination basin of the reprocessing system of FIG. 1;

FIG. 3 depicts a cross-sectional side view of proximal and distal portions of an endoscope that may be decontaminated using the reprocessing system of FIG. 1;

FIG. 4A depicts a top portion of alternative schematic diagram of the single decontamination basin of the reprocessing system of FIG. 1;

FIG. 4B depicts a bottom portion of the alternative schematic diagram; and

FIG. 5 depicts a variation of a magnified portion of FIG. 4A.

### MODES OF CARRYING OUT THE INVENTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values±10% of the recited value, e.g. “about 90%” may refer to the range of values from 81% to 99%. In addition, as used herein, the terms “patient,” “host,” “user,” and “subject” refer to any human or animal subject and are not intended to limit the systems or methods to human use, although use of the subject invention in a human patient represents a preferred embodiment.

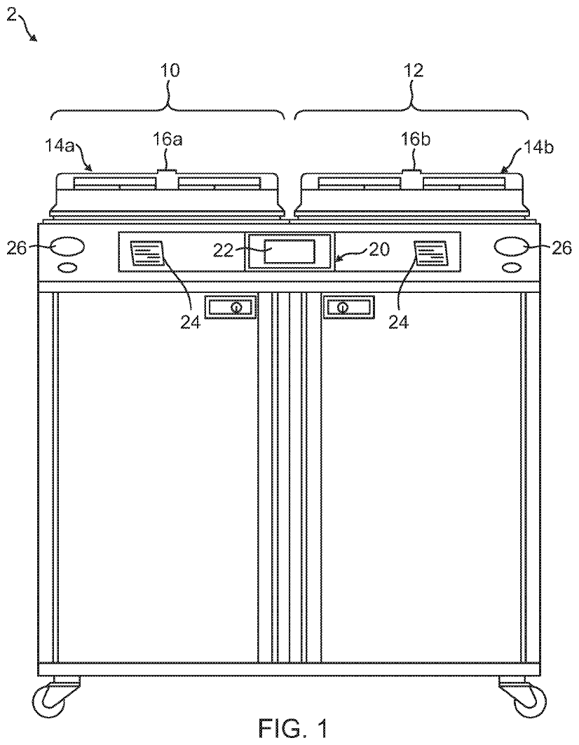

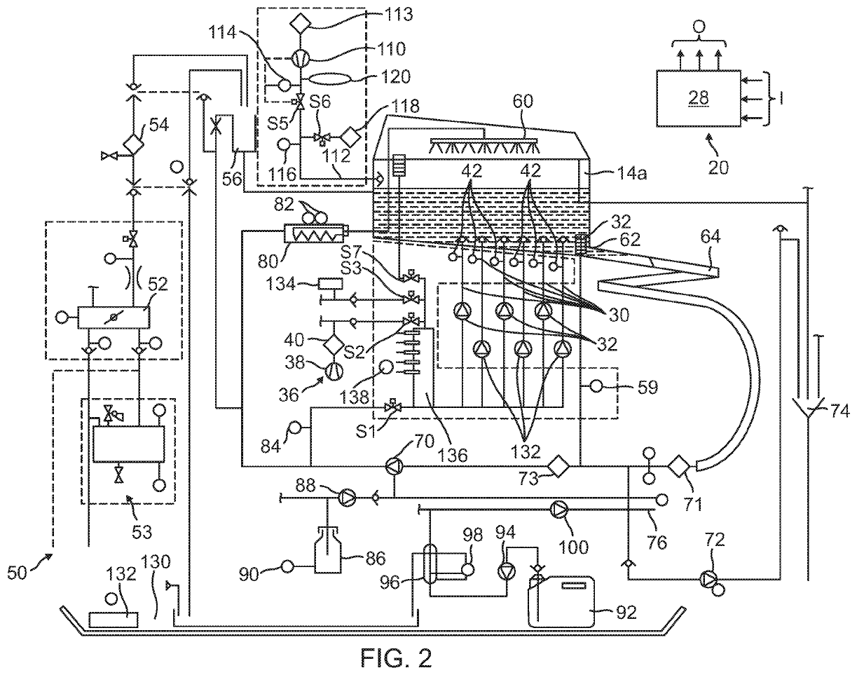

FIGS. 1-2 show an exemplary reprocessing system 2 that may be used to decontaminate endoscopes and other medical devices that include channels or lumens formed therethrough. System 2 of this example generally includes a first station 10 and a second station 12. Stations 10, 12 are at least substantially similar in all respects to provide for the decontamination of two different medical devices simultaneously or in series. First and second decontamination basins 14a, 14b receive the contaminated devices. Each basin 14a, 14b is selectively sealed by a respective lid 16a, 16b. In the present example, lids 16a, 16b cooperate with respective basins 14a, 14b to provide a microbe-blocking relationship to prevent the entrance of environmental microbes into basins 14a, 14b during decontamination operations. By way of example only, lids 16a, 16b may include a microbe removal or HEPA air filter formed therein for venting. Lids 16a, 16b may also contain reprocessing fluids (e.g., washing fluids and decontamination fluids) and delivery conduits for the fluids.

A control system 20 includes one or more microcontrollers, such as a programmable logic controller PLC, for controlling decontamination and user interface operations. Although one control system 20 is shown herein as controlling both decontamination stations 10, 12, those skilled in the art will recognize that each station 10, 12 can include a dedicated control system. A visual display 22 displays decontamination parameters and machine conditions for an operator, and at least one printer 24 prints a hard copy output of the decontamination parameters for a record to be filed or attached to the decontaminated device or its storage packaging. It should be understood that printer 24 is merely optional. In some versions, visual display 22 is combined with a touch screen input device. In addition, or in the alternative, a keypad and/or other user input feature is provided for input of decontamination process parameters and for machine control. Other visual gauges 26 such as pressure meters and the like provide digital or analog output of decontamination or medical device leak testing data.

FIG. 2 diagrammatically illustrates just one decontamination station 10 of reprocessing system 2, but those skilled in the art will recognize that decontamination station 12 may be configured and operable just like decontamination station 10. It should also be understood that reprocessing system 2 may be provided with just one single decontamination station 10, 12 or more than two decontamination stations 10, 12.

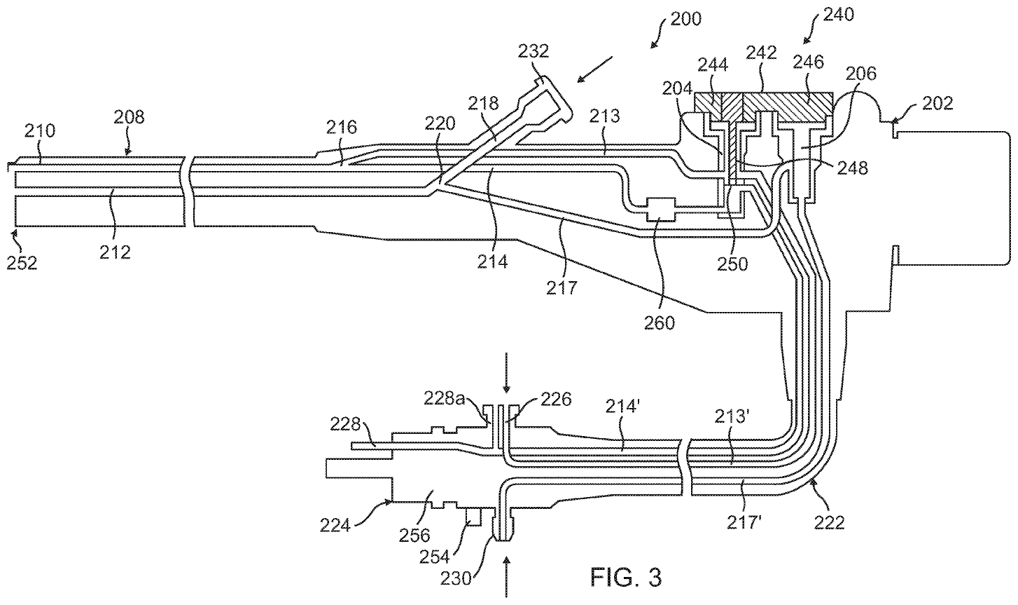

Decontamination basin 14a receives an instrument having at least one lumen or channel, e.g., endoscope 200 (FIG. 3), therein for decontamination. Any internal channels of endoscope 200 are connected with flush conduits, such as flush lines 30. Each flush line 30 is connected to an outlet of a corresponding pump 32, such that each flush line 30 has a dedicated pump 32 in this example. Pumps 32 of the present example comprise peristaltic pumps that pump fluid, such as liquid and air, through the flush lines 30 and any internal channels of endoscope 200. Alternatively, any other suitable kind of pumps may be used. In the present example, pumps 32 can either draw liquid from basin 14a through a filtered drain and a valve S1; or draw decontaminated air from an air supply system 36 through a valve S2. Air supply system 36 of the present example includes a pump 38 and a microbe removal air filter 40 that filters microbes from an incoming air stream. Typically, a single endoscope 200 may be received in basin 14a, however, in some embodiments, sufficient flush lines 30 are provided to basin 14a such that two endoscopes 200 may be received therein and connected to flush lines 30.

A pressure switch or sensor 42 is in fluid communication with each flush line 30 for sensing excessive pressure in the flush line. Any excessive pressure or lack of flow sensed may be indicative of a partial or complete blockage e.g., by bodily tissue or dried bodily fluids in an endoscope 200 channel to which the relevant flush line 30 is connected. The isolation of each flush line 30 relative to the other flush lines 30 allows the particular blocked channel to be easily identified and isolated, depending upon which sensor 42 senses excessive pressure or lack of flow. Pressure sensor 42 may also help protect endoscope 200 from damage due to excessive fluid pressurization because when excess pressure is detected the pumps 32 may be deactivated.

Basin 14a is in fluid communication with a water source 50, such as a utility or tap water connection including hot and cold inlets, and a mixing valve 52 flowing into a break tank 56. A microbe removal filter 54, such as a 0.2 μm or smaller absolute pore size filter, decontaminates the incoming water, which is delivered into break tank 56 through the air gap to prevent backflow. A sensor 59 monitors liquid levels within basin 14a. An optional water heater 53 can be provided if an appropriate source of hot water is not available. The condition of filter 54 can be monitored by directly monitoring the flow rate of water therethrough or indirectly by monitoring the basin fill time using a float switch or the like. When the flow rate drops below a select threshold, this indicates a partially clogged filter element that requires replacement.

A basin drain 62 drains liquid from basin 14a through an enlarged helical tube 64 into which elongated portions of endoscope 200 can be inserted. Drain 62 is in fluid communication with a recirculation pump 70 and a drain pump 72. Recirculation pump 70 recirculates liquid from basin drain 62 to a spray nozzle assembly 60, which sprays the liquid into basin 14a and onto endoscope 200. A coarse screen 71 and a fine screen 73 filter out particles in the recirculating fluid. Drain pump 72 pumps liquid from basin drain 62 to a utility drain 74. A level sensor 76 monitors the flow of liquid from pump 72 to utility drain 74. Pumps 70, 72 can be simultaneously operated such that liquid is sprayed into basin 14a while basin 14a is being drained, to encourage the flow of residue out of basin 14a and off of endoscope 200. Of course, a single pump and a valve assembly could replace dual pumps 70, 72.

An inline heater 80 with temperature sensors 82, upstream of recirculation pump 70, heats the liquid to optimum temperatures for cleaning and/or disinfection. A pressure switch or sensor 84 measures pressure downstream of circulation pump 70. In some variations, a flow sensor is used instead of pressure sensor 84, to measure fluid flow downstream of circulation pump 70. Detergent solution 86 is metered into the flow downstream of circulation pump 70 via a metering pump 88. A float switch 90 indicates the level of detergent 86 available. Decontaminant solution 92 is metered into the flow upstream of circulation pump 70 via a metering pump 94. To more accurately meter decontaminant solution 92, pump 94 fills a metering pre-chamber 96 under control of a fluid level switch 98 and control system 20. By way of example only, decontaminant solution 92 may comprise a disinfectant or a liquid chemical sterilant. An exemplary disinfectant may comprise activated glutaraldehyde solution, such as CIDEX® Activated Glutaraldehyde Solution by Advanced Sterilization Products of Irvine, Calif. or ortho-phthalaldehyde OPA, such as CIDEX® ortho-phthalaldeyde solution by Advanced Sterilization Products of Irvine, Calif. Exemplary liquid-chemical sterilants may include peracetic acid (“PAA”) or hydrogen peroxide.

Some endoscopes 200 include a flexible outer housing or sheath surrounding the individual tubular members and the like that form the interior channels and other parts of endoscope 200. This housing defines a closed interior space, which is isolated from patient tissues and fluids during medical procedures. It may be important that the sheath be maintained intact, without cuts or other holes that would allow contamination of the interior space beneath the sheath. Therefore, reprocessing system 2 of the present example includes means for testing the integrity of such a sheath. In particular, an air pump e.g., pump 38 or another pump 110 pressurizes the interior space defined by the sheath of endoscope 200 through a conduit 112 and a valve S5. In the present example, a HEPA or other microbe-removing filter 113 removes microbes from the pressurizing air. A pressure regulator 114 prevents accidental over pressurization of the sheath. Upon full pressurization, valve S5 is closed and a pressure sensor 116 looks for a drop in pressure in conduit 112, which would indicate the escape of air through the sheath of endoscope 200. A valve S6 selectively vents conduit 112 and the sheath of endoscope 200 through an optional filter 118 when the testing procedure is complete. An air buffer 120 smoothes out pulsation of pressure from air pump 110. During reprocessing, endoscope 200 may be maintained in a pressurized state to prevent undesired ingress of reprocessing fluids in the body of the endoscope.

In the present example, each station 10, 12 also contains a drip basin 130 and spill sensor 132 to alert the operator to potential leaks. An alcohol supply 134, controlled by a valve S3, can supply alcohol to channel pumps 32 after rinsing steps, to assist in removing water from channels 210, 212, 213, 214, 217, 218 of endoscope 200.

Flow rates in lines 30 can be monitored via channel pumps 32 and pressure sensors 42. If one of pressure sensors 42 detects too high a pressure, the associated pump 32 is deactivated. The flow rate of pump 32 and its activated duration time provide a reasonable indication of the flow rate in an associated line 30. These flow rates are monitored during the process to check for blockages in any of the channels of endoscope 200. Alternatively, the decay in the pressure from the time pump 32 cycles off can also be used to estimate the flow rate, with faster decay rates being associated with higher flow rates.

A more accurate measurement of flow rate in an individual channel may be desirable to detect subtler blockages or malfunctions (e.g., pump malfunction) to ensure that the correct flow rates through each channel is achieved. To that end, a metering tube 136 having a plurality of level indicating sensors 138 fluidly connects to the inputs of channel pumps 32. In some versions, a reference connection is provided at a low point in metering tube 136 and a plurality of sensors 138 are arranged vertically above the reference connection. By passing a current from the reference point through the fluid to sensors 138, it can be determined which sensors 138 are immersed and therefore determine the level within metering tube 136. In addition, or in the alternative, any other suitable components and techniques may be used to sense fluid levels. By shutting valve S1 and opening a vent valve S7, channel pumps 32 draw exclusively from metering tube 136. The amount of fluid being drawn can be very accurately determined based upon sensors 138. By running each channel pump 32 in isolation, the flow therethrough can be accurately determined based upon the time and the volume of fluid emptied from metering tube 136.

In addition to the input and output devices described above, all of the electrical and electromechanical devices shown are operatively connected to and controlled by control system 20. Specifically, and without limitation, switches and sensors 42, 59, 76, 84, 90, 98, 114, 116, 132 136 provide input I to microcontroller 28, which controls the cleaning and/or disinfection cycles and other machine operations in accordance therewith. For example, microcontroller 28 includes outputs O that are operatively connected to pumps 32, 38, 70, 72, 88, 94, 100, 110, valves S1, S2, S3, S5, S6, S7, and heater 80 to control these devices for effective cleaning and/or disinfection cycles and other operations.

As shown in FIG. 3, endoscope 200 has a head part (control body) 202. Head part 202 includes openings 204, 206 formed therein. During normal use of endoscope 200, an air/water valve not shown and a suction valve not shown are arranged in openings 204, 206. A flexible shaft (umbilical tube) 208 is attached to head part 202. A combined air/water channel 210 and a combined suction/biopsy channel 212 are accommodated in shaft 208. A separate air channel 213 and water channel 214 are also arranged in head part 202 and merge into air/water channel 210 at the location of a joining point 216. It will be appreciated that the term “joining point” as used herein refers to an intersecting junction rather than being limited to a geometrical point and, the terms may be used interchangeably. Furthermore, a separate suction channel 217 and biopsy channel 218 are accommodated in head part 202 and merge into suction/biopsy channel 212 at the location of a joining point 220. Although endoscope 200 is shown with four independent channels (i.e., air channel 213, water channel 214, suction channel 217 and biopsy channel 218) that overlap into two combined channels (i.e., air/water channel 210 and suction/biopsy channel 212), commercially available endoscopes may have fewer channels or more channels, e.g., between one channel and eight channels. These channels may be used for different purposes. Typically, for a given endoscope, these channels each have a uniform diameter along their lengths. The lengths are dependent upon the overall length of the endoscope and whether the channel exists through the entire endoscope, such that the length of the channel equals or is substantially equal to the length of the endoscope, or only in a portion of it (e.g., between the control body and the distal end of the umbilical tube, such that the length of the channel equals or is substantially equal to the length of the umbilical tube).

An endoscope may include various channels. Diameters and lengths of these channels are also provided based on an exemplary endoscope that is approximately 3.5 meters long. It should be appreciated however, that these dimensions, particularly the lengths, may vary based on the length of the endoscope. The air channel may be used to deliver air to clear debris from the endoscope, such as a lens of the endoscope. An exemplary air channel may have a diameter of approximately 1.2 mm and comprise a first segment having a length of approximately 1700 mm and a second segment having a length of approximately 1400 mm. The suction channel may be used to aspirate fluids and debris that is directly connected thereto. An exemplary suction channel may have a diameter of approximately 1.2 mm and comprise a first segment having a length of approximately 1700 mm and a second segment having a length of approximately 1400 mm. The biopsy channel may be used to provide an entry point and passageway to the instrument channel of the endoscope. An exemplary biopsy channel may have a diameter of approximately 4.2 mm and a length of approximately 50 mm. The instrument channel may be used to provide a passageway to the distal end of the endoscope from the biopsy channel for forceps or another instrument to collect a biopsy tissue sample. An exemplary instrument channel may have a diameter of approximately 3.8 mm a length of approximately 1700 mm Commonly, the instrument channel and biopsy channel may be collectively referred to as the biopsy channel, e.g., as in this application. The water-jet channel or auxiliary-water channel may be used to deliver a jet stream of sterile fluid to wash away debris on the tissue or blood that may be blocking the view of the treatment site. An exemplary water-jet channel may have a diameter of approximately 1.0 mm and comprise a length of approximately 3500 mm. The balloon channel may be used to aspirate air fluid to fill a balloon cover that lays over the endoscope's insertion tube close to the distal tip of the endoscope to keep the field of view intact within the lumen of the GI tract. An exemplary balloon channel may have a diameter of approximately 0.8 mm and a length of approximately 2400 mm and a second segment having a length of approximately 1400 mm Finally, the endoscope may also include an elevator channel to house a wire connected to an elevator mechanism. The wire may be manipulated to change the orientation of the elevator mechanism, which may be used to angulate forceps or other instruments at the distal tip for purposes of, e.g., endoscopic retrograde cholangiopancreatographic biopsies. An exemplary suction channel may have a diameter of approximately 0.8 mm a length of approximately 1660 mm and a second segment having a length of approximately 1400 mm. Further, as set forth above, some of these channels may join with others. Commonly, the water and air channels may join and the biopsy and suction channels may join.

In head part 202, air channel 213 and water channel 214 open into opening 204 for the air/water valve not shown. Suction channel 217 opens into opening 206 for the suction valve not shown. Furthermore, a flexible feed hose 222 connects to head part 202 and accommodates channels 213′, 214′, 217′, which are connected to air channel 213, water channel 214, and suction channel 217 via respective openings 204, 206. In practice, feed hose 222 may also be referred to as the light-conductor casing. The mutually connecting air channels 213, 213′ will collectively be referred to below as air channel 213. The mutually connecting water channels 214, 214′ will collectively be referred to below as water channel 214. The mutually connecting suction channels 217, 217′ will collectively be referred to below as suction channel 217. A connection 226 for air channel 213, connections 228, 228a for water channel 214, and a connection 230 for suction channel 217 are arranged on the end section 224 also referred to as the light conductor connector of flexible hose 222. When the connection 226 is in use, connection 228a is closed off. A connection 232 for biopsy channel 218 is arranged on head part 202.

A channel separator 240 is shown inserted into openings 204, 206. Channel separator 240 comprises a body 242 and plug members 244, 246, which occlude respective openings 204, 206. A coaxial insert 248 on plug member 244 extends inwardly of opening 204 and terminates in an annular flange 250, which occludes a portion of opening 204 to separate channel 213 from channel 214. By connecting lines 30 to openings 226, 228, 228a, 230, 232, liquid for cleaning and disinfection can be flowed through endoscope channels 213, 214, 217, 218 and out of a distal tip 252 of endoscope 200 via channels 210, 212. Channel separator 240 ensures that such liquid flows all the way through endoscope 200 without leaking out of openings 204, 206; and isolates channels 213, 214 from each other so that each channel 213, 214 has its own independent flow path. One of skill in the art will appreciate that various endoscopes having differing arrangements of channels and openings may require modifications to channel separator 240 to accommodate such differences while occluding ports in head 202 and keeping channels separated from each other so that each channel can be flushed independently of the other channels. Otherwise, a blockage in one channel might merely redirect flow to a connected unblocked channel.

A leakage port 254 on end section 224 leads into an interior portion 256 of endoscope 200 and is used to check for the physical integrity thereof, namely to ensure that no leakage has formed between any of the channels and the interior 256 or from the exterior to the interior 256.

Successful decontamination of an endoscope, such as endoscope 200, requires providing various decontamination liquids, such as water, detergent, disinfectant, and sterilant, or decontamination fluids (which include decontamination liquids and various gases, e.g., air or nitrogen) to all surfaces, including the inner surfaces of all of the endoscope's channels, i.e., air channel 213, water channel 214, suction channel 217, biopsy channel 218, air/water channel 210, and suction/biopsy channel 212, in sufficient volume for sufficient time. Particular challenges in endoscope reprocessing arise because endoscopes typically include between two to eight channels or lumens having small but different diameters (e.g., from approximately 0.5 millimeters to approximately 10 millimeters), which may be from approximately three meters to six meters long, and which may merge together (e.g., channels 210 and 212) or separate from each other depending on the direction of flow.

For a single uniform channel having a laminar circular cross-section, laminar fluid flow may be theoretically described according to the he Hagen-Poiseuille equation:

$$Δ P = \frac{8 μ L Q}{π {R}^{4}} ,$$

where ΔP is the change in pressure in the channel, μ is the viscosity of the fluid, L is the length of the channel, Q is the volume flow rate of the channel, and R is the radius of the channel. The Hagen-Poiseuille equation provides that a drop in pressure of a fluid flowing through a long pipe of uniform cross section is proportional to the length of the pipe and inversely proportional to the radius to the fourth power. As detailed above, however, endoscope channels have geometry that is somewhat more complicated than a single pipe of a uniform cross section. For example, in endoscope 200, the channels bend and merge, and, as seen in FIG. 3, may include segments, such as segment 260, that have different dimensions than other portions of a given channel. Furthermore, turbulent flow may be desired because it has been found that turbulent flows clean better than laminar flows. Even in such cases, it is instructive to note that resistance varies as a function of length and radius.

Due to the differences in the diameters and lengths of each channel and the complexity of their geometry, which includes certain channels joining or merging together to form combined channels, challenges exist in providing each of the decontamination fluids to all of the interior surfaces of the channels simultaneously in sufficient volume and for sufficient time to achieve a desired decontamination goal, such as cleaning, rinsing, disinfecting, sterilizing, or drying. For example, if fluid were to be provided to one large-diameter channel and one small-diameter channel, the fluid might not readily flow through the small-diameter channel because the resistance to flow would be greater in the smaller channel than the larger channel. An additional challenge arises from certain channels joining together. Consider, for example, suction channel 217, biopsy channel 218, and the combined suction/biopsy channel 212. Reliable delivery of fluids to the inner surfaces of these three channels may be assisted by simultaneously introducing fluid through connections 230 and 232 under pressures whereby all fluids introduced through both connections readily flow at desired volume flow rates toward distal end 252. Thus, undesired stagnation or backwards flow should be avoided. Stagnation or backward flow toward connection 230 could result if, for example, the inlet pressure at joining point 220 is equal to or greater than the pressure somewhere in suction channel 217 between connection 230 and joining point 220.

Conventional systems typically provide decontamination fluids from a single source operating at a single pressure to each of the channels separately, for example, one or two channels at a time, and perhaps with assistance of devices such as channel separator 240. Thus, the overall time required to clean, decontaminate, and dry an endoscope may be reduced by overcoming the challenges set forth above in a way that permits simultaneous delivery of fluids to a greater number of the endoscope's channels, such as three or more, including all of them, without compromising, e.g., the volume of fluid to be delivered to each channel, the volume flow rate of each fluid in each channel, and overall delivery time to deliver fluid to each channel. Thus, for example, for an endoscope that includes eight channels, substantial time savings may be achieved if a decontamination fluid could be delivered to all eight channels simultaneously while providing it in sufficient volumes and at sufficient volume flowrates to achieve cleaning, decontamination, and drying goals for each channel.

Applicant has determined that each inlet pressure, i.e., the pressure at a location where fluid is introduced into or first enters a channel (e.g., connections 226, 228, 230, and 232, as well as joining points 216 and 220) may be tailored such that a decontamination fluid may simultaneously flow through each of the channels, ultimately toward distal end 252, within a range of desired flow rates. That is, the pressures at some or all of connections 226, 228, 230, and 232 should be different to account for pressure drop as a function of length and diameter and the pressure at each joining point.

For example, assume that a decontamination fluid is simultaneously supplied at equal pressure to suction channel 217 via connection 230 and to biopsy channel 218 via connection 232. Because biopsy channel 218 has a larger diameter and shorter length than suction channel 217, the pressure drop between connection 230 and joining point 220 should be greater than the pressure drop from connection 232 to joining point 220. Accordingly, the pressure at joining point 220 may be greater than the pressure in suction channel 217 at a location between connection 230 and joining point 220. Such may cause flow in suction channel 217 to stagnate or move within suction channel 217 toward connection 230. These pitfalls may be avoided, however, by providing fluid at connection 232 at a lower pressure than at connection 230. Preferably, the pressures at connections 232 and 230 are chosen such that the pressure in channel 218 just before joining point 220 is equal to the pressure in channel 217 just before joining point 220 such that the fluid in both channels may proceed past joining point 220 and into biopsy/suction channel 212 at that pressure and without slowing the flow through either of the channels 217 and 218. Further, the pressure at joining point 220 should be sufficient to force fluid through biopsy/suction channel 212 and out of distal end 252.

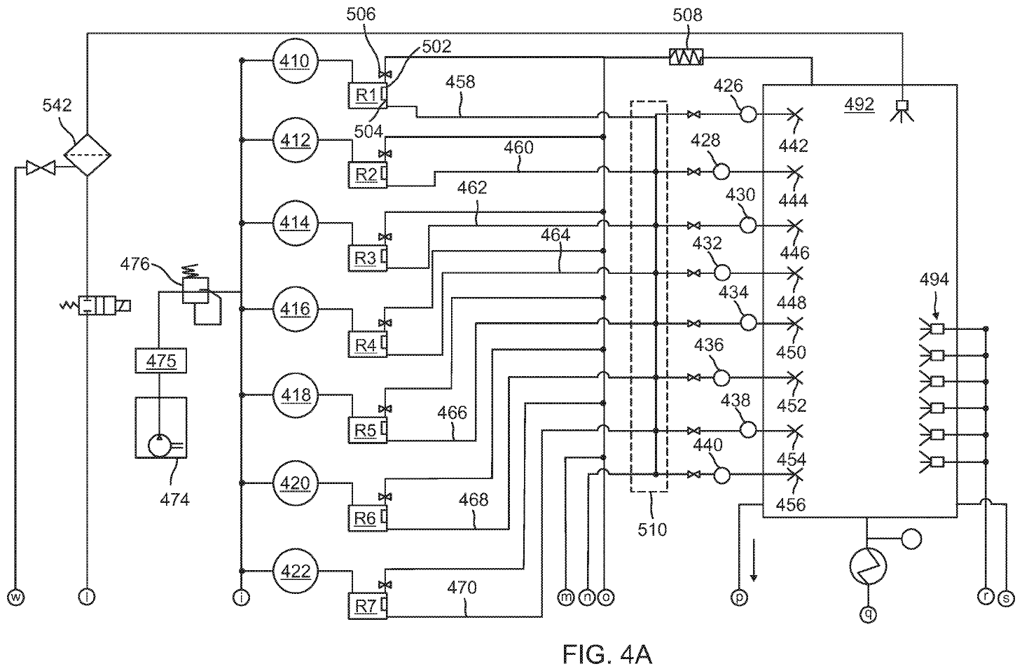

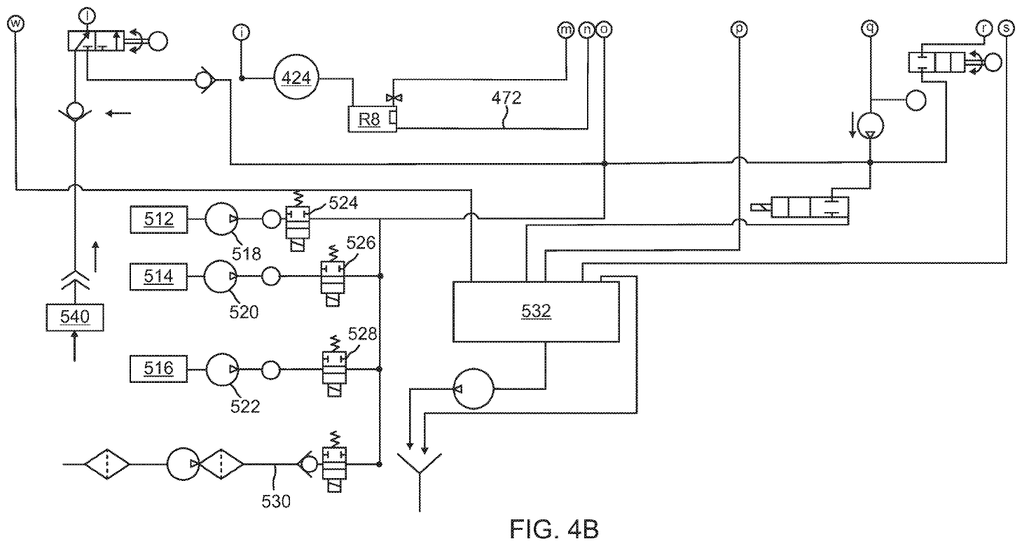

FIGS. 4A and 4B together reflect an alternative schematic concerning the function of reprocessing system 2 for a single decontamination station 10. A key difference between the schematic of FIGS. 4A and 4B and the schematic of FIG. 2 is that the schematic of FIGS. 4A and 4B includes at least one variable orifice device, such as variable-orifice device 410, which may assist in tailoring input pressures of decontamination fluids for the reasons described above. As shown, the system includes eight variable-orifice devices 410, 412, 414, 416, 418, 420, 422, and 424, such that this system may be used to reprocess endoscopes having up to eight channels. At least one sensor for measuring a flow characteristic, e.g., flow rate, pressure, or shear stress, may be disposed on each flush line. For example, a flow sensor, e.g., flow sensor 426, may be disposed on a flush line, e.g., 458, between one of the variable-orifice devices and a corresponding output from the system, e.g., output 442, that connects to a corresponding channel of the endoscope. As shown, eight flush lines are provided, i.e., 458, 460, 462, 464, 466, 468, 470, and 472; eight flow sensors are provided, i.e., 426, 428, 430, 432, 434, 436, 438, and 440; and eight outputs are provided, i.e., 442, 444, 446, 448, 450, 452, 454, and 456. These outputs may be provided in a washing chamber 492, which may include nozzles 494 for spraying decontamination fluids into the chamber.

The system may also include eight reservoirs or sources R1-R8, one upstream of each flush line. These sources may be filled with decontamination fluids, particularly decontamination liquids, such that when full, the liquid may be ejected therefrom via gas supplied by the compressed-gas source 474, which may optionally flow through an air filter 475. As such, each reservoir may include level sensors, e.g., a minimum-height level sensor and a maximum-height level sensor, which may be used to activate and deactivate the corresponding air-proportional valve such that the compressed gas may begin to force out the liquid when the reservoir is full and such that the compressed gas flow may be stopped when the reservoir becomes empty. For example, minimum-height and maximum height level sensors of R1 are labeled as 502 and 504, respectively. Valves, such as valve 506 just downstream of R1, may also be provided to prevent back flow through the reservoirs. An inline heater 508 may also be provided to heat liquid entering the reservoirs to a desired temperature suitable for the liquid and the procedure being conducted. A manifold 510 may optionally be included, e.g., to provide for facilitated mounting of various flow-characteristic sensors in the system, such as pressure sensors.

The system may also include one or more sources 512, 514, and 516 of decontamination fluids, e.g., peracetic acid, detergent, and alcohol, which may be refilled by a user. Pumps 518, 520, and 522 may pump these decontamination fluids through the system, e.g., to R1-R8, when corresponding solenoids 524, 526, and 528 are in an open state. An optional gas purge line 530, with structures such as a pressurized source of a gas (e.g., air), a filter, and a solenoid, may also be provided for purging the decontamination fluids from at least a portion of the system. Water may be provided to the system via a water source 540, which may be filtered by filter 542. Other components and features of the system that are useful for commercial versions of a reprocessing system, but are not a focus of the claimed subject matter, include, e.g., drain sump 532 and other heaters, pumps, solenoids, flow-characteristic sensors, valves, etc, which are reflected in FIGS. 4A and 4B, but not provided with reference numerals.

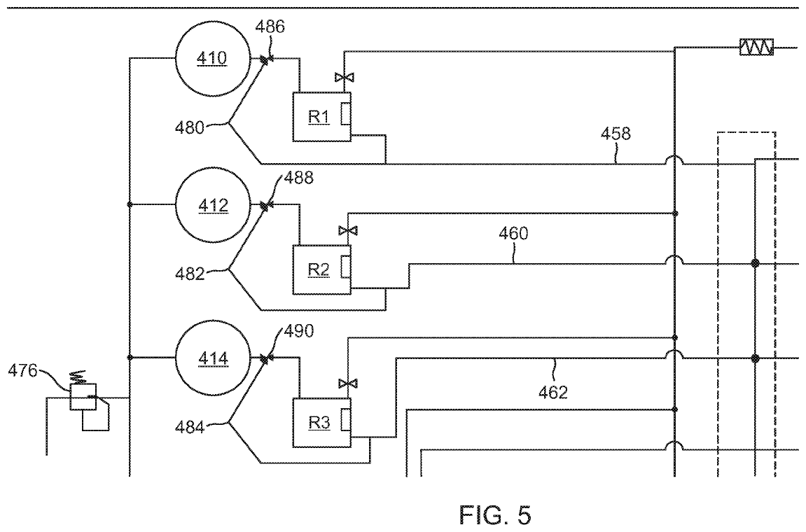

As explained herein, a gas, e.g., air, may be used to introduce, propel, and purge liquids from the flush lines (e.g., 458) and channels of an endoscope connected to the flush lines. When a gas, e.g., air is used to purge cleaning liquids from the flush lines and endoscope channels, the gas may flow through a source (e.g., R1) containing little or no cleaning liquid, such as a volume having a height less than a height that would be detected by a minimum-height level sensor, such as sensor 504 of R1. Alternatively, as reflected in FIG. 5, the gas may flow through bypass lines, e.g., 480, 482, and 484 such that it does not flow through a source, e.g., R1, R2, and R3, which may contain residual liquid. As shown, the bypass lines (e.g., 480), like the sources (e.g., R1), may be connected to a corresponding variable-orifice device (e.g., 410) and a flush line (e.g., 458), such that a fluid, typically a gas, e.g., air, may flow therethrough from the corresponding variable-orifice device to the corresponding flush line. Accordingly, valves 486, 488, and 490 may be disposed between a corresponding variable-orifice device e.g., 410, 412, and 414, on one side and a source, e.g., R1, R2, and R3, and a bypass line, 480, 482, and 484, on the other side to direct the gas from the variable-orifice device toward the source or toward the bypass line to have the gas bypass the source before reaching the corresponding flush line. Although the example provided in FIG. 5 reflects components corresponding only to flush lines 458, 460, and 462, further iterations of the components described in this paragraph may be included on the remaining flush lines 464, 466, 468, 470, and 472. Thus, for example, a bypass line may be disposed between each flush line and each corresponding variable-orifice device shown in FIGS. 4A and 4B.

Gases provide particular challenges for delivering them at predetermined flow rates and for a sufficient amount of time to purge disinfection liquids from an endoscope or to dry all of the internal surfaces each of the endoscope's channels because gases are compressible and sensitive to pressure differences throughout the endoscope. The system described herein may overcome these challenges such that it may be used for simultaneously delivering gas to at least two channels of an endoscope in a manner that facilitates and shortens the amount of time required to purge or dry these channels. Further, because each channel of the endoscope should be dried after introducing one or more liquids and before introducing another liquid, substantial time savings may be achieved by purging or drying these channels simultaneously instead of sequentially. For example, in an exemplary decontamination procedure, the endoscope may be decontaminated by providing the decontamination fluids to each channel according to the following sequence: water to rinse, air to dry, detergent to wash, water to rinse, air to dry, disinfectant to decontaminate, water to rinse, air to dry, alcohol to clean, and then air to dry. In an exemplary sterilization procedure, the endoscope may be sterilized by providing the decontamination fluids to each channel according to a similar sequence, but instead of providing a disinfectant, a liquid-chemical sterilant and then a neutralizer may be provided.

As such, the variable-orifice devices, e.g., device 410, may be closed loop or open loop air-proportional valves. Various acceptable air-proportional valves are manufactured and sold by companies, such as ASCO, a division of Emerson, SMC corporation. Air may be provided to the endoscope channels simultaneously from a centralized compressed gas source, e.g., compressor 474. The variable-orifice devices may thus regulate the pressure of gas received at each inlet of each endoscope channel to inlet pressures that have been previously determined (or predetermined) to correspond to predetermined flow rates through each channel of the endoscope, and additionally or alternatively corresponding to shear stresses along the internal surfaces of these channels. Alternatively, each of the variable-orifice devices may be configured to receive inputs from flow sensors connected to other channels such that each of the variable-orifice devices maintains equal or approximately equal flow rates (e.g., each of the flow rates are within 10% of the other) through each of the channels. For example, for an endoscope, such as endoscope 200, which has an air channel and a water channel that merge and a biopsy channel and a suction channel that merge, providing air having a temperature of approximately room temperature to each of connections 226, 228, 230, and 232 (FIG. 3) at pressures of between approximately 10 psi and 30 psi, such as 20 psi, for between 10 seconds to 10 minutes, such as thirty seconds, one minute, two minutes, or five minutes, should result in flow rates that should dry the entirety of the internal surfaces of the endoscope's channels by the dual mechanisms of purging liquid from the distal end of the endoscope while evaporating any droplets that remain within the channels. As used herein, the term drying means that residual liquid on a surface has a thickness of less than approximately 0.06 mm. Accordingly, the variable orifice of each of the variable-orifice devices may be sized to drop the pressure from the pressure output by compressor 474 (which may be between approximately 50 psi and approximately 70 psi, such as approximately 60 psi) to the desired input pressure at each of the connections 226, 228, 230, and 232. Additionally, a pressure regulator 476 may be included between compressor 474 and the variable-orifice devices to regulate the pressure to be between approximately 10 psi and approximately 35 psi, e.g., between approximately 14 psi and approximately 28 psi, which may assist the variable-orifice devices in further regulating the pressures.

Ideally, the size of each variable orifice may be changed before and during a decontamination procedure. For example, by use of a user-input device, e.g., a keyboard or touch screen, connected to control system 20 (FIG. 2), a user may set the desired orifice sizes based on the predetermined input pressures for any endoscope to be reprocessed by the system. Furthermore, by comparing the flow rates measured at each of the flow sensors to predetermined flow rates desired for drying the channels, the pressures at each of the outputs, e.g., output 442, can be modified automatically by control system 20 when the flow rate measured by the flow sensors (e.g., 426) differs from a predetermined flow rate in a corresponding endoscope channel. For example, when the flow rate at the flow sensor is less than a predetermined flow rate, the variable orifice of the variable-orifice device may be expanded. Conversely, when the flow rate at the flow sensor is greater than the predetermined flow rate, the variable orifice of one or more of the variable-orifice devices may be constricted. Additionally or alternatively, and as noted above, each of the variable-orifice devices may be configured to receive inputs from flow sensors connected to other channels such that each of the variable-orifice devices maintains equal or approximately equal flow rates (e.g., each of the flow rates are within about 10% or about 20% of the other) through each of the channels. The flow rates within each of the channels may change based on the location and size of any liquid droplets within the channels that remains to be dried or ejected from the distal end of the endoscope.

Computational fluid dynamics simulations were conducted on a gastrointestinal endoscope having a suction channel and biopsy channel that combine into a suction/biopsy channel. In the simulation, the suction channel was specified as having a length of 1.7 meters and a diameter of 4 millimeters, the biopsy channel was specified as having a length of 0.022 meters and a diameter of 6 millimeters, and the suction/biopsy channel was specified as having a length of 2.178 meters and a diameter of 6 millimeters. Further inputs of inlet air pressures to the suction channel and biopsy channel, as well as outputs of shear stresses, flow rates, and flow regime are provided in Table 1. The simulation assumed simultaneous and steady-state flow of air through each channel. As reflected in Table 1, for an inlet pressure of 28 psi to the suction channel, by lowering the inlet pressure to the biopsy channel from 28 psi to 14 psi. the flow rate in the suction channel can be increased by over 300%, from 1.21 m/s to 3.83 m/s and the shear stress in the suction channel can be increased by nearly 600%, from 9 pascals to 53 pascals. Further, because the flow rates and shear stresses in the channels are a function of the inlet pressures to the channels, the flow rates and shear stresses in the channels may be adjusted to approximate each other. For example, in Table 1, when the biopsy-channel inlet is 14 psi and the suction-channel inlet pressure is 28 psi, the flow rates are, respectively, 4.4 m/s and 3.83 m/s, and the shear stresses are, respectively, 59 pascals and 53 pascals. As such, the inlet pressures to the biopsy channel and suction channel may be chosen to optimize procedure time against flow rates and corresponding shear stresses within each channel.

| Inlet | Inlet | Flow | Flow | Shear | Shear | | |
|---|---|---|---|---|---|---|---|
| Pressure | Pressure | Rate | Rate | Stress | Stress | Flow | Flow |
| Biopsy | Suction | Biopsy | Suction | Biopsy | Suction | Regime | Regime |
| (psig) | (psig) | (m/s) | (m/s) | (Pa) | (Pa) | Biopsy | Suction |
| 28 | 28 | 6.22 | 1.21 | 115 | 9 | Turbulent | Turbulent |
| 21 | 28 | 5.42 | 2.66 | 85 | 30 | Turbulent | Turbulent |
| 14 | 28 | 4.4 | 3.83 | 59 | 53 | Turbulent | Turbulent |

By virtue of the embodiments illustrated and described herein, Applicant has devised a method and variations thereof for using decontamination system to drive a gas, e.g., air, to advance a liquid through channels of an endoscope, purge the channels of the liquid, and dry the channels. A first volume of a gas, e.g., air may be flowed through a first variable-orifice device (e.g., 410) and into a first flush line (e.g., 458), which may contain a decontamination liquid or the gas. A second volume of the gas may also be flowed through a second variable-orifice device (e.g., 412) and into a second flush line (e.g., 460), which may also contain the decontamination liquid or the gas. An endoscope may be connected to the flush lines. For example, a first channel of the endoscope may be connected to a first output of the first flush line and a second channel of the endoscope may be connected to a second output of the second flush line. As such, the method also includes flowing the first volume of air through the first channel and flowing the second volume of air through the second channel. In preferred applications of this method, the first channel of the endoscope and the second channel of the endoscope merge into a first joined channel. In these applications, the first volume of the gas and the second volume of the gas may both be flowed through the first joined channel.

One or more flow-characteristic sensors (e.g., flow-rate sensors 426, 428) may be disposed along the first flush line, second flush line, or both. As such, the method may also include steps of measuring a flow characteristic, e.g., flow rate, along the first flush line, the second flush line, or both. Based on this measurement or these measurements, a first size of a first orifice of the first variable-orifice device may be changed. Additionally or alternatively, based on this measurement or these measurements, a second size of a second orifice of the second variable-orifice device may be changed. A reason for changing the first size and the second size is to cause the flow-rate characteristics measured in the first flush channel and the second flush channel to become at least approximately equal, e.g., equal. As such, after the first size or second size has been changed, the method may include a step of measuring a first subsequent flow characteristic in the first flush line and measuring a second subsequent flow characteristic in the second line and comparing them to each other to determine whether they are at least approximately equal to each other.

To assist in conducting the foregoing, a controller may be included in the decontamination system that is configured to receive the flow characteristic from the flow-characteristic sensor and to send an instruction based on the flow characteristic. As such, further steps of the method may include receiving at the controller the flow characteristic, determining, with the controller, whether the flow characteristic should be changed, and sending an instruction to the first variable-orifice, the second variable-orifice device, or both, to change the size of the first orifice, the second orifice, or both. Accordingly, the step of changing the first size and the second size may occur after the controller has sent the instruction to change the size of the first orifice, the second orifice, or both.

Any of the examples or embodiments described herein may include various other features in addition to or in lieu of those described above. The teachings, expressions, embodiments, examples, etc., described herein should not be viewed in isolation relative to each other. Various suitable ways in which the teachings herein may be combined should be clear to those skilled in the art in view of the teachings herein.

Having shown and described exemplary embodiments of the subject matter contained herein, further adaptations of the methods and systems described herein may be accomplished by appropriate modifications without departing from the scope of the claims. In addition, where methods and steps described above indicate certain events occurring in certain order, it is intended that certain steps do not have to be performed in the order described but in any order as long as the steps allow the embodiments to function for their intended purposes. Therefore, to the extent there are variations of the invention, which are within the spirit of the disclosure or equivalent to the inventions found in the claims, it is the intent that this patent will cover those variations as well. Some such modifications should be apparent to those skilled in the art. For instance, the examples, embodiments, geometrics, materials, dimensions, ratios, steps, and the like discussed above are illustrative. Accordingly, the claims should not be limited to the specific details of structure and operation set forth in the written description and drawings.

1. A decontamination system, comprising:
a first source of a first decontamination fluid,
a gas compressor;
a first variable-orifice device;
a second variable-orifice device;
a first flush line connected to the gas compressor via the first variable-orifice device and further connected to the first source of the first decontamination fluid, the first flush line including a first output; and
a second flush line connected to the gas compressor via the second variable-orifice device and further connected to the first source of the first decontamination fluid, the second flush line including a second output.

2. The decontamination system of claim 1, wherein the first variable-orifice device includes a first orifice having a first size and the second variable-orifice device includes a second orifice having a second size that is different than the first size.

3. The decontamination system of claim 2, further comprising:
a first flow-characteristic sensor disposed along the first flush line between the first variable-orifice device and the first output; and
a second flow-characteristic sensor disposed along the second flush line between the first variable-orifice device and the second output.

4. The decontamination system of claim 3, wherein the first flow-characteristic sensor comprises a first flow-rate sensor.

5. The decontamination system of claim 3, wherein the first source is disposed between the first variable-orifice device and the first flush line.

6. The decontamination system of claim 4, further comprising a second source for containing a second decontamination fluid, the second source disposed between the second variable-orifice device and the second flush line.

7. The decontamination system of claim 6, wherein the second decontamination fluid is the same as the first decontamination fluid.

8. The decontamination system of claim 6, further comprising a first bypass line connected to the first variable-orifice device and the first flush line.

9. The decontamination system of claim 8, further comprising a second bypass line connected to the second variable-orifice device and the second flush line.

10. The decontamination system of claim 9, further comprising a first valve connected to the first variable-orifice device, the first flush line, and the first bypass line, the first valve configured to direct air from the first variable-orifice device toward the first source or toward the bypass line.

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. The decontamination system of claim 10, wherein the first output is connected to a first channel of an endoscope.

25. The decontamination system of claim 24, wherein the second output is connected to a second channel of the endoscope.

26. (canceled)

27. (canceled)

28. (canceled)

29. (canceled)

30. The decontamination system of claim 10, wherein the first channel and the second channel merge at a joining point into a joined channel.

31. (canceled)

32. (canceled)

33. (canceled)

34. A method of operating a decontamination system, comprising:
flowing a first volume of a gas through a first variable-orifice device;
flowing the first volume of the gas into a first flush line containing a decontamination liquid, the first flush line comprising a first output;
flowing a second volume of the gas through a second variable-orifice device;
flowing the second volume of the gas into a second flush line containing the decontamination liquid, the second flush line comprising a second output.

35. The method of claim 34, wherein the gas comprises air.

36. The method of claim 34, further comprising:
connecting a first channel of an endoscope to the first output;
connecting a second channel of the endoscope to the second output;
flowing the first volume of the gas through the first channel; and
flowing the second volume of the gas through the second channel.

37. The method of claim 36, wherein the first channel and the second channel merge into a joined channel.

38. (canceled)

39. The method of claim 36, wherein the first variable-orifice device includes a first orifice having a first size and the second variable-orifice device includes a second orifice having a second size.

40. The method of claim 39, further comprising measuring a flow rate with a flow sensor along the first flush line or the second flush line.

41. (canceled)

42. (canceled)

43. The method of claim 40, further comprising changing the first size, the second size, or both the first size and the second size, based on the flow rate such that a first subsequent flow rate in the first channel and a second subsequent flow rate in the second channel become equal or approximately equal.

44-51. (canceled)
